SEQUENCE LISTING (5 sequences)

SEQ ID NO: 1 - protein, 620 aa, Drosophila melanogaster
MAAGVFKSFMRDFFAVKYDEQRNDPQAERLDGNGRLYPNCSSDVWLRSCEREIVDPIEGHHSGHIPKWICGSLLRNGPGSWKVGDMTFGHLFDCSALLHRFAIRNGRVTYQNRFVDTETLRKNRSAQRIVVTEFGTAAVPDPCHSIFDRFAAIFRPDSGTDNSMISIYPFGDQYYTFTETPFMHRINPCTLATEARICTTDFVGVVNHTSHPHVLPSGTVYNLGTTMTRSGPAYTILSFPHGEQMFEDAHVVATLPCRWKLHPGYMHTFGLTDHYFVIVEQPLSVSLTEYIKAQLGGQNLSACLKWFEDRPTLFHLIDRVSGKLVQTYESEAFFYLHIINCFERDGHVVVDICSYRNPEMINCMYLEAIANMQTNPNYATLFRGRPLRFVLPLGTIPPASIAKRGLVKSFSLAGLSAPQVSRTMKHSVSQYADITYMPTNGKQATAGEESPKRDAKRGRYEEENLVNLVTMEGSQAEAFQGTNGIQLRPEMLCDWGCETPRIYYERYMGKNYRYFYAISSDVDAVNPGTLIKVDVWNKSCLTWCEENVYPSEPIFVPSPDPKSEDDGVILASMVLGGLNDRYVGLIVLCAKTMTELGRCDFHTNGPVPKCLHGWFAPNAI

SEQ ID NO: 2 - DNA, 1863 bp, Artificial Sequence
  Yarrowia codon-optimized DmNinaB
atggccgctggtgttttcaagtcttttatgcgagatttctttgctgttaagtacgatgagcagcgaaacgacccccaggccgagcgactggacggcaacggacgactgtaccccaactgctcctctgatgtttggcttcgatcttgcgagcgagagatcgttgaccccattgagggccaccactccggtcacattcccaagtggatttgcggttccctgctccgaaacggccccggctcttggaaggttggcgacatgaccttcggccacctgttcgactgctccgccctgctccaccgatttgccattcgaaacggacgagtcacctaccagaaccgatttgttgacactgagactctgcgaaagaaccgatctgcccagcgaattgttgtcaccgagtttggcactgccgctgttcccgatccctgtcactccatcttcgaccgatttgccgccatttttcgacccgattctggaaccgataactccatgatttccatctaccccttcggcgaccagtactacactttcaccgagactccctttatgcaccgaattaacccctgcactctcgctactgaggctcgaatctgcaccaccgacttcgttggcgttgtcaaccacacttctcacccccacgttcttccctctggcactgtttacaacctgggcaccactatgacccgatctggacccgcttacactatcctctctttcccccacggcgagcagatgttcgaggacgctcacgttgtcgccactctgccctgccgatggaagctgcaccccggttatatgcacaccttcggcctcactgaccactactttgtcattgttgagcagcccctttccgtttccctcactgagtacatcaaggcccagcttggcggacagaacctttccgcttgcctcaagtggttcgaggaccgacccactctctttcaccttattgatcgagtttccggcaagctggtccagacctacgagtccgaggctttcttctacctgcacatcatcaactgctttgagcgagatggccacgttgtcgttgacatttgctcttaccgaaaccccgagatgattaactgcatgtacctggaggccattgccaacatgcagactaaccccaactacgctaccctctttcgaggacgaccccttcgattcgtcctgcccctcggcactattccccccgcctctatcgccaagcgaggactcgtcaagtccttctccctcgctggactctccgctccccaggtttctcgaaccatgaagcactccgtttctcagtacgccgatattacctacatgcccaccaacggaaagcaggccactgctggagaggagtcccccaagcgagatgccaagcgaggccgatacgaggaggagaaccttgtcaacctggttactatggagggctctcaggccgaggcttttcagggcaccaacggcattcagcttcgacccgagatgctgtgtgattggggctgtgagactccccgaatctactacgagcgatacatgggcaagaactaccgatacttctacgccatttcttccgatgttgatgctgtcaaccccggcaccctcatcaaggttgatgtctggaacaagtcttgtcttacctggtgcgaggagaacgtctacccctctgagcccatttttgtcccctctcccgatcccaagtccgaggacgatggcgttatcctggcctctatggttcttggcggtcttaacgaccgatacgtcggccttattgttctttgtgccaagaccatgaccgagctgggccgatgtgatttccacaccaacggacccgttcccaagtgcctccacggttggtttgctcccaacgccatttag

SEQ ID NO: 3 - DNA, 7690 bp, Artificial Sequence
  plasmid, concatenation of 3 sequences
cctgctagcgtcgacggccattatggccgagccctaaatctgtgttatactgttgaaatgtgttgattccaccaaacgacattaagaaccaactattgcttgaactttttttgatggcttagcagatttgaactattaagaaggacttctccagtattgtatatattgaagaaaaccatctgaagttgcaacatttgaatttttcctatgacgtttttttttcctcgcttcatcaatatttttcagttttccatgttgaggccataatctgggttaactcgaattgacaagtatatagtcaagctggaggtatgaagtacgtaacatttaaagagcattgagacgatattctgttcatgataactataattttaaatatgaagttacgctaattgcaaagtagcaaaaaatttggacgagtccggaatcgaaccggagacctctcccatgctaagggagcgcgctaccgactacgccacacgcccatttcttattgtaatttctagtcactgtaaaaagtgaaatcagtttaaaatgaaagtgtctatcaaaacttattatccactatcaagtaattactcatgactagttttggtaccgttcgtataatgtatgctatacgaagttataagctttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagcatgcttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatggcatccattgcctccgcgaccggctgtagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtttgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacaaggcaagctaagcttataacttcgtataatgtatgctatacgaacggtaaccggtgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaggatccattattatttgaattcgcgggggatctcccatgtctctactggtggtggtgcttctttggaattattggaaggtaaggaattgccaggtgttgctttcttatccgaaaagaaataaattgaattgaattgaaatcgtagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgagagctctgtcggaagaggaaccacctaccctctatagtctagcatccatcttattacatatacgatgtagaaatatgacataaaggtaaagattggaaagctgccatcaaatttaatgggggtggaacgcacgacttgataatgcaataggataatgagtgacaacatataaagtggaacgagaaaccataatattattatgaagaatcatcgatattgtccaaattgtattgttatggaaatggtattcaacaactatctcaaaagtcacctatttctcgtgcttttcgcattctatcacctgtattattatatttcatcaaaaagatgaatcatccaatgtaaatgacacacaaatgtgcaagtgccaagctattaagtggaataatggccttgttatttaatggtaagagccttctgaggccaatctgccttcacgtacacaaccaccttgttaggaacaataataccaacatagtttggccgtgatggcctaggaggtagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

SEQ ID NO: 4 - DNA, 8580 bp, Artificial Sequence
  plasmid, concatenation of 2 sequences
cctgctagcgtcgacggccattatggccgagccctaaatctgtgttatactgttgaaatgtgttgattccaccaaacgacattaagaaccaactattgcttgaactttttttgatggcttagcagatttgaactattaagaaggacttctccagtattgtatatattgaagaaaaccatctgaagttgcaacatttgaatttttcctatgacgtttttttttcctcgcttcatcaatatttttcagttttccatgttgaggccataatctgggttaactcgaattgacaagtatatagtcaagctggaggtatgaagtacgtaacatttaaagagcattgagacgatattctgttcatgataactataattttaaatatgaagttacgctaattgcaaagtagcaaaaaatttggacgagtccggaatcgaaccggagacctctcccatgctaagggagcgcgctaccgactacgccacacgcccatttcttattgtaatttctagtcactgtaaaaagtgaaatcagtttaaaatgaaagtgtctatcaaaacttattatccactatcaagtaattactcatgactagttttggtaccgttcgtataatgtatgctatacgaagttataagctttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagcatgcttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatggcatccattgcctccgcgaccggctgtagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtttgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacaaggcaagctaagcttataacttcgtataatgtatgctatacgaacggtaaccggtgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaggatccatggccccatccattcgtaagttctttgctggtggtgtctgtagaaccaacgtccaattgccaggtaaggttgtcgtcatcactggtgccaacactggtatcggtaaggaaaccgccagagaattggcctcccgtggtgccagagtctatattgcctgtcgtgacgttttgaagggtgaatccgctgcttccgaaattcgtgttgataccaagaactcccaagtcttggtccgtaagttggacttgtccgacactaagtctatccgtgctttcgctgaaggtttcttggccgaagaaaagcaattgcacatcttgattaacaacgctggtgttatgatgtgtccttactctaagactgctgatggtttcgaaacccacttgggtgtcaaccacttgggtcacttcttgttgacctacttgttgttggaacgtttgaaagtctccgctccagccagagtcgttaacgtttcctccgtcgcccatcacatcggtaagatcccattccacgacttgcaatccgaaaagcgttactcccgtggttttgcttactgccactccaagttggccaacgtcttgtttacccgtgaattggccaagcgtttgcaaggtactggtgtcaccacctacgccgttcacccaggtgtcgtccgttccgaattggtccgtcactcctccttgttgtgcttgttgtggcgtttgttctccccattcgtcaaaaccgccagagaaggtgcccaaacctccttgcactgtgccttggccgaaggtttggaacctttgtccggtaagtacttctctgactgcaagcgtacctgggtttcccctagagccagaaacaacaagactgccgaacgtttgtggaacgtttcctgcgaattgttgggtattcgttgggaataagaattcgcgggggatctcccatgtctctactggtggtggtgcttctttggaattattggaaggtaaggaattgccaggtgttgctttcttatccgaaaagaaataaattgaattgaattgaaatcgtagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgagagctctgtcggaagaggaaccacctaccctctatagtctagcatccatcttattacatatacgatgtagaaatatgacataaaggtaaagattggaaagctgccatcaaatttaatgggggtggaacgcacgacttgataatgcaataggataatgagtgacaacatataaagtggaacgagaaaccataatattattatgaagaatcatcgatattgtccaaattgtattgttatggaaatggtattcaacaactatctcaaaagtcacctatttctcgtgcttttcgcattctatcacctgtattattatatttcatcaaaaagatgaatcatccaatgtaaatgacacacaaatgtgcaagtgccaagctattaagtggaataatggccttgttatttaatggtaagagccttctgaggccaatctgccttcacgtacacaaccaccttgttaggaacaataataccaacatagtttggccgtgatggcctaggaggtagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

SEQ ID NO: 5 - DNA, 9543 bp, Artificial Sequence
  plasmid, concatenation of 2 sequences
cctgctagcgtcgacggccattatggccgagccctaaatctgtgttatactgttgaaatgtgttgattccaccaaacgacattaagaaccaactattgcttgaactttttttgatggcttagcagatttgaactattaagaaggacttctccagtattgtatatattgaagaaaaccatctgaagttgcaacatttgaatttttcctatgacgtttttttttcctcgcttcatcaatatttttcagttttccatgttgaggccataatctgggttaactcgaattgacaagtatatagtcaagctggaggtatgaagtacgtaacatttaaagagcattgagacgatattctgttcatgataactataattttaaatatgaagttacgctaattgcaaagtagcaaaaaatttggacgagtccggaatcgaaccggagacctctcccatgctaagggagcgcgctaccgactacgccacacgcccatttcttattgtaatttctagtcactgtaaaaagtgaaatcagtttaaaatgaaagtgtctatcaaaacttattatccactatcaagtaattactcatgactagttttggtaccgttcgtataatgtatgctatacgaagttataagctttcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagcatgcttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatggcatccattgcctccgcgaccggctgtagaacagcgggcagttcggtttcaggcaggtcttgcaacgtgacaccctgtgcacggcgggagatgcaataggtcaggctctcgctgaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtttgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacaaggcaagctaagcttataacttcgtataatgtatgctatacgaacggtaaccggtgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaggatccatggctgccggtgtcttcaagtcttttatgcgtgacttcttcgctgtcaaatacgacgaacaacgtaacgatccacaagccgaacgtttggacggtaacggtcgtttgtacccaaactgctcctccgatgtctggttgcgttcctgcgaacgtgaaatcgttgatccaatcgaaggtcaccactccggtcacatccctaagtggatttgcggttccttgttgcgtaacggtcctggttcctggaaggtcggtgacatgaccttcggtcacttgttcgactgctccgccttgttgcacagattcgccattcgtaacggtcgtgtcacctaccaaaaccgtttcgttgacaccgaaaccttgcgtaagaaccgttccgcccaacgtatcgtcgtcaccgaatttggtactgctgctgtcccagatccatgccactccatcttcgatagattcgccgctattttccgtccagattccggtactgacaactccatgatttccatttatccattcggtgaccaatactatactttcaccgaaaccccatttatgcatagaattaacccatgcactttggccaccgaagccagaatctgcaccaccgacttcgtcggtgtcgtcaaccacacttcccaccctcatgtcttgccttccggtactgtctacaacttgggtactaccatgaccagatctggtccagcctacactattttgtccttcccacacggtgaacaaatgttcgaagacgctcatgtcgtcgccaccttgccatgcagatggaagttgcacccaggttatatgcacaccttcggtttgaccgaccattacttcgttatcgtcgaacaaccattgtctgtctccttgaccgaatacattaaggcccaattgggtggtcaaaatttgtctgcctgtttgaagtggttcgaagaccgtccaaccttattccacttgattgatcgtgtctccggtaagttggtccaaacctacgaatccgaagccttcttctacttgcacatcatcaactgtttcgaacgtgacggtcacgtcgtcgttgacatctgttcctaccgtaaccctgaaatgatcaactgcatgtacttggaagccatcgctaatatgcaaaccaatccaaactatgctaccttgttccgtggtagacctttgagattcgtcttgccattgggtactatccctccagcctccatcgccaagcgtggtttggtcaagtccttctccttggctggtttatccgctccacaagtctctcgtaccatgaagcactccgtttcccaatacgctgatatcacttacatgcctaccaatggtaagcaagccactgctggtgaagaatccccaaagcgtgatgctaagcgtggtcgttacgaagaagaaaacttagtcaacttggttaccatggaaggttcccaagccgaagccttccaaggtactaatggtatccaattgcgtccagaaatgttgtgcgattggggttgcgaaactcctagaatctactatgaacgttacatgggtaagaactaccgttacttctacgccatctcttccgacgttgatgctgtcaacccaggtactttgatcaaggtcgatgtctggaataagtcctgtttgacctggtgcgaagaaaacgtctacccttccgaacctatctttgtcccttcccctgacccaaagtccgaagacgacggtgtcatcttggcctccatggtcttgggtggtttgaacgaccgttacgtcggtttgatcgttttgtgcgctaaaaccatgaccgaattgggtagatgcgatttccataccaatggtcctgtccctaagtgcttgcatggttggtttgctcctaacgccatttaagaattcgcgggggatctcccatgtctctactggtggtggtgcttctttggaattattggaaggtaaggaattgccaggtgttgctttcttatccgaaaagaaataaattgaattgaattgaaatcgtagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgagagctctgtcggaagaggaaccacctaccctctatagtctagcatccatcttattacatatacgatgtagaaatatgacataaaggtaaagattggaaagctgccatcaaatttaatgggggtggaacgcacgacttgataatgcaataggataatgagtgacaacatataaagtggaacgagaaaccataatattattatgaagaatcatcgatattgtccaaattgtattgttatggaaatggtattcaacaactatctcaaaagtcacctatttctcgtgcttttcgcattctatcacctgtattattatatttcatcaaaaagatgaatcatccaatgtaaatgacacacaaatgtgcaagtgccaagctattaagtggaataatggccttgttatttaatggtaagagccttctgaggccaatctgccttcacgtacacaaccaccttgttaggaacaataataccaacatagtttggccgtgatggcctaggaggtagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagct

(19) United States
(12) Patent Application Publication
(10) Pub. No.: US 2022/0356503 A1
(43) Pub. Date: Nov. 10, 2022

(54) NOVEL BETA-CAROTENE OXIDASES

(71) Applicant: DSM IP Assets B.V., Heerlen (NL)

(72) Inventors: Peter Louis HOUSTON, Kaiseraugst (CH); René Marcel DE JONG, Kaiseraugst (CH)

(21) Appl. No.: 17/626,946

(22) PCT Filed: Jul. 15, 2020
(86) PCT No.: PCT/EP2020/069933

§ 371 Date: Jan. 13, 2022

Related U.S. Application Data
(60) Provisional application No. 62/874,765, filed on Jul. 16, 2019.

(30) Foreign Application Priority Data
Oct. 29, 2019 (EP) 19205961.6

(51) Int. Cl. C12P 23/00; C12N 9/02; C12N 1/16; C12N 15/81

(52) U.S. Cl. CPC C12P 23/00; C12Y 113/11063; C12N 9/0069; C12N 1/16; C12N 15/81; C12N 2800/102

(57) ABSTRACT

The present invention is related to a method for increasing the trans-specificity of a beta-carotene oxidase (BCO), particularly insect BCO, to be used in the production of vitamin A aldehyde (retinal) from conversion of beta-carotene, with at least about 75 to 100% of retinal in the trans-isoform.

The present invention is related to a method for increasing the trans-specificity of a beta-carotene oxidase (BCO), particularly insect BCO, to be used in the production of vitamin A aldehyde (retinal) from conversion of beta-carotene, with at least about 78 to 100% of retinal in the trans-isoform.

Retinal is an important intermediate/precursor in the process of retinoid production, in particular such as vitamin A production. Retinoids, including vitamin A, are one of very important and indispensable nutrient factors for human beings which have to be supplied via nutrition. Retinoids promote well-being of humans, inter alia in respect of vision, the immune system and growth.

Current chemical production methods for retinoids, including vitamin A and precursors thereof, have some undesirable characteristics such as e.g. high-energy consumption, complicated purification steps and/or undesirable by-products. Therefore, over the past decades, other approaches to manufacture retinoids, including vitamin A and precursors thereof, including microbial conversion steps, which would be more economical as well as ecological, have been investigated.

In general, the biological systems that produce retinoids are industrially intractable and/or produce the compounds at such low levels that commercial scale isolation is not practicable. There are several reasons for this, including instability of the retinoids in such biological systems or the relatively high production of by-products. Instability of vitamin A can be circumvented by producing acetylated forms, such as e.g. retinyl or vitamin A acetate. Since retinoids are chiral compounds, they occur in both trans- and cis-form. For industrial purpose, however, the trans-isoforms, i.e. trans-retinyl acetate, are the most important forms.

Starting from beta-carotene, the first step in such biological process for production of vitamin A/vitamin A acetate, mainly in trans-isoform, is catalyzed by BCO, leading to two units of retinal. From the known BCO enzymes, insect BCOs are of particular interest due to their high enzymatic activity, i.e. nearly complete conversion of beta-carotene into retinal (up to 95% conversion). However, they are not fully trans-specific, meaning they produce a certain level of cis-retinal, which cannot be converted to trans-retinol acetate (VitA acetate) anymore. This results in a loss of carbon flux to the desired trans retinyl acetate product.

Thus, it is an ongoing task to improve productivity and/or selectivity or specificity of the enzymatic conversion of beta-carotene into vitamin A including improvement of the first enzymatic step, i.e. enzymatic conversion of beta-carotene into trans-retinal. Particularly, it is desirable to optimize a high-activity enzyme such as known from insects via increasing the trans-specificity leading to mainly trans-retinal which is furthermore converted into trans retinyl acetate.

Surprisingly, we now found that modification of certain amino acids in beta-carotene oxidases, particularly insect BCOs showing both trans- and cis-activity, can boost the formation of trans-retinal, i.e. increase the stereo-selectivity without any substantive compromise on productivity of the BCOs, leading to conversion ratios in the range of at least about 78% of trans-isoforms based on total retinoids including retinal present/produced in the respective host cell.

Thus, the present invention is directed to modified (trans-selective) BCO enzymes, particularly insect enzyme, which can be expressed in a suitable host cell, such as a carotenoid/retinoid-producing host cell, particularly fungal host cell, with the activity of catalyzing the conversion of beta-carotene into trans-retinal, with a percentage of trans-retinal in the range of at least about 78%, such as e.g. about 80, 85, 90, 92, 95, 96, 97, 98, 99 or even 100% based on total retinoids present in/produced by the host cell. Preferably, the non-modified BCO enzymes which are to be modified according to the present invention are originated from Drosophila, such as e.g. D. melanogaster. Particularly, the activity of the modified BCOs, i.e. conversion of beta-carotene into retinal, is in the range of at least about 5, 10, 15, 20, 30, 40, 50, 60, 70, 80, 90, 95 to about 100%, i.e. about the same as the respective non-modified BCO.

Particularly, the present invention is directed to modified BCO enzymes, preferably modified insect BCO, more preferably originated from Drosophila, such as e.g. D. melanogaster, i.e. modified BCO comprising one or more modification(s), i.e. amino acid substitution(s), preferably comprising one or more amino acid substitution(s) in a sequence with at least about 60%, such as e.g. 65, 70, 75, 80, 85, 90, 92, 95, 97, 98, 99% or up to 100% identity to SEQ ID NO:1, wherein the one or more amino acid substitution(s) are located at position(s) corresponding to amino acid residue(s) selected from position 91 and/or 499 in a polypeptide according to SEQ ID NO:1.

The terms modified “beta-carotene oxidase”, “beta-carotene oxidizing enzyme”, “beta-carotene oxygenase”, “enzyme having beta-carotene oxidizing activity” or “BCO” are used interchangeably herein and refer to beta-carotene 15,15′-dioxygenase enzymes (EC 1.13.11.63), sometimes also referred to as beta-carotene 15,15′-monooxygenase enzymes (EC 1.14.99.36), which are capable of catalyzing the conversion of beta-carotene into two units of retinal, with at least about 78 to 100% as trans-retinal and with a total conversion rate (i.e. enzyme activity) of at least about 5, 10, 15, 20, 30, 40, 50, 60, 70, 80, 90, 95 to about 100%, i.e. wherein at least about 5% beta-carotene are converted into retinal. Such modified BCOs are referred herein as trans-selective enzymes. A preferred modified isoform is a polypeptide with at least 60% identity to SEQ ID NO:1 comprising one or more amino acid substitution(s) on one or more position(s) as defined herein.

The terms “conversion”, “enzymatic conversion”, “oxidation”, “enzymatic oxidation”, or “cleavage” in connection with enzymatic catalysis of beta-carotene are used interchangeably herein and refer to the action of the modified BCOs as defined herein.

As used herein, the terms “stereoselective”, “selective”, “trans-selective” enzyme with regards to modified BCO are used interchangeably herein. They refer to enzymes, i.e. modified BCOs as disclosed herein, with increased catalytic activity towards trans-isomers, i.e. increased activity towards catalysis of beta-carotene into trans-retinal. A modified enzyme according to the present invention is trans-specific, if the percentage of trans-isoforms, such as e.g. trans-retinal, is in the range of at least about 78% based on total retinoids including retinal produced by such a modified enzyme or such carotene-producing host cell, particularly fungal host cell, comprising and expressing such modified enzyme.

The term “conversion ratio” refers to the percentage of trans-forms, i.e. a ratio of trans-forms present in a mix comprising cis- and trans-forms of a compound, particularly the ratio of trans-forms of retinoids including trans-retinal, to total retinoids including retinal as e.g. present in the respective host cell, wherein the trans-selectivity is resulting from action of the modified BCO enzymes as of the present invention.

As used herein, the term “fungal host cell” includes particularly yeast as host cell, such as e.g. Yarrowia or Saccharomyces.

The modified enzymes as defined herein might be introduced into a suitable host cell, i.e. expressed as heterologous enzyme in a carotenoid-producing host cell, particularly fungal host cell, or might be used in isolated form (i.e. in a cell-free system). Preferably, the enzymes as described herein are introduced and expressed as heterologous enzymes in a suitable host cell, such as e.g. a carotenoid-producing host cell, particularly fungal host cell, as described in the art.

Suitable BCO enzymes which might be used to generate the modified BCOs according to the present invention, might be obtained from any beta-carotene/retinol-producing source, such as e.g. plants, animals, including humans, algae, fungi, including yeast, or bacteria, preferably from insects with a relatively high percentage of cis-selectivity as defined herein, such as BCOs with a cis-selectivity of about 22% or less, i.e. capable of catalyzing the conversion of beta-carotene into retinal with a percentage of 22% or less cis-retinal based on total retinoids. Particular useful BCO enzymes can be obtained from Drosophila, i.e. D. melanogaster, such as e.g. DmNinaB (according to SEQ ID NO:1), or as exemplified in Table 5. These suitable enzymes, particularly insect enzymes, to be used for the present invention, are capable of converting beta-carotene to at least about 5, 10, 15, 20, 30, 40, 50, 60, 70, 80, 90, 95 to about 100% of retinal.

A particular difference of the retinoid cycle between insects and vertebrates underscores the efficiency and regulation of optical pigment presentation as cis-retinal into rhodopsin. These differences led to the evolution of DmNinaB and other insect enzymes that explains why all of these enzymes make more cis isoforms. The vertebrate system regulates the specificity of the cis isoform of retinol by final promotion of trans-retinol to 11-cis by which is catalyzed by reduction to retinol and subsequent oxidation and isomerization by the Rpe65 enzyme. In contrary, the insect cis-retinal presentation system directly employs the cis-isoforms made by DmNinaB without modification. In summary, insects have a direct way to promote the cis-retinal to integration into rhodopsin indicating that we can expect that all insect enzymes make cis retinoids similar to DmNinaB.

Particularly, suitable insect BCO enzymes to be modified as described herein, can be recognized in the protein sequence databases by a partial amino acid sequence of at least 5 amino acid residues selected from [GWP]-C-E-[TIME]-P, preferably G-C-E-T-P, corresponding to position 496 to 500 in the polypeptide according to SEQ ID NO:1 (all motifs in Prosite syntax, as defined in (https://prosite.expasy.org/scanprosite/scanprosite_doc.html) which includes the position T499 that can be mutated as described herein to increase trans-selectivity of the insect BCO. Further insect BCO enzymes comprising this conserved motif and which are suitable for performance of the present invention, i.e. introduction of amino acid substitutions as defined herein, can be identified in a BLAST search (see e.g. Table 5).

In one embodiment, the modified BCO enzyme as defined herein comprises an amino acid substitution at a position corresponding to residue 91 in the polypeptide according to SEQ ID NO:1 leading to tryptophan or phenylalanine at said residue, such as e.g. via substitution of leucine by tryptophan (L91W) or leucine by phenylalanine (L91F). Said modified enzyme might be originated from Drosophila, such as Drosophila melanogaster. The use of such modified enzyme for oxidation of beta-carotene comprising one of the above-mentioned mutations leads to conversion ratios in the range of at least about 78 to 91%, i.e. at least about 78 to 91% of retinoids including retinal are in trans-form, whereby the activity of the enzyme, i.e. conversion of beta-carotene into retinal, will stay about the same as for the respective non-mutated BCO, such as in the range of about 20%.

In another embodiment, the modified BCO enzyme as defined herein comprises an amino acid substitution at a position corresponding to residue 499 in the polypeptide according to SEQ ID NO:1 leading to leucine, methionine or isoleucine at said residue, such as e.g. via substitution of threonine by leucine (T499L), threonine by methionine (T499M) or threonine by isoleucine (T499I). Said modified enzyme might be originated from Drosophila, such as Drosophila melanogaster. The use of such modified enzyme for oxidation of beta-carotene comprising one of the above-mentioned mutations leads to conversion ratios in the range of at least about 83 to 95%, i.e. at least about 83 to 95% of retinoids including retinal are in trans-form, whereby the activity of the enzyme, i.e. conversion of beta-carotene into retinal, will stay about the same.

In one particular embodiment, the modified BCO enzyme as defined herein comprises a combination of amino acid substitutions at positions corresponding to residue 91 and 499 in the polypeptide according to SEQ ID NO:1 leading to tryptophan or phenylalanine at position 91 and leucine, methionine or isoleucine at position 499, preferably leucine at position 499, such as e.g. via substitution of leucine by tryptophan (L91W) or phenylalanine (L91F) combined with substitution of threonine by leucine (T499L), methionine (T499M) or isoleucine (T499I). Most preferred are combinations L91W-T499L or L91F-T499L. Said modified enzymes might be originated from Drosophila, such as Drosophila melanogaster. The use of such modified enzymes for oxidation of beta-carotene comprising one of the above-mentioned combined mutations leads to conversion ratios in the range of at least about 92 to 97%, i.e. at least about 92 to 97% of retinoids including retinal are in trans-form, whereby the activity of the enzyme, i.e. conversion of beta-carotene into retinal, will stay about the same, such as in the range of at least about 5 to about 10%, as compared to the respective BCO without said double mutations.

Using one of the modified BCO enzymes as defined herein, an increase of at least about 7%, such as in the range of about 7 to 33% and more, in the conversion rate, i.e. in production of trans-isomers in the mix of total retinoids including retinal can be achieved via enzymatic conversion of beta-carotene as compared to the amount of trans-isoforms using of a non-modified BCO according to SEQ ID NO:1, whereby the activity of the enzyme, i.e. conversion of beta-carotene into retinal, will stay about the same, i.e. in the range of about at least 5%.

The host cell as described herein is capable of conversion of beta-carotene into trans-retinal with conversion ratios of at least about 78%, such as e.g. 80, 85, 90, 92, 95, 96, 97, 98, 99 or even 100% (based on total retinoids including retinal produced by said host cell) towards generation of trans isoforms, while showing (maintaining) high activity towards conversion of beta-carotene into retinal, i.e. in the range of about at least 5%.

The host cell might be further modified, i.e. producing more copies of genes and/or proteins, such as e.g. more copies of modified BCOs with selectivity towards formation of trans-retinal as defined herein. This may include the use of strong promoters, suitable transcriptional- and/or translational enhancers, or the introduction of one or more gene copies into the carotenoid-producing host cell, particularly fungal cells, leading to increased accumulation of the respective enzymes in a given time. The skilled person knows which techniques to use depending on the host cell. The increase—as well as reduction—of gene expression can be measured by various methods, such as e.g. Northern, Southern or Western blot technology as known in the art.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

Thus, the present invention is directed to a carotenoid-producing host cell, particularly fungal host cell, as described herein comprising an expression vector or a polynucleotide encoding modified BCO as described herein which has been integrated in the chromosomal DNA of the host cell. Such carotenoid-producing host cell comprising a heterologous polynucleotide either on an expression vector or integrated into the chromosomal DNA encoding BCOs as described herein is called a recombinant host cell. The carotenoid-producing host cell, particularly fungal host cell, might contain one or more copies of a gene encoding the modified BCO enzymes, as defined herein, such as e.g. polynucleotides encoding polypeptides with at least about 60% identity to SEQ ID NO:1 comprising one or more amino acid substitution(s) as defined herein, leading to overexpression of such genes encoding said modified BCO enzymes, as defined herein.

Based on the sequences as disclosed herein and on the preference for trans-isoforms, i.e. conversion ratios in the range of at least about 78 to 100% towards formation of trans-retinoids including trans-retinal, one could easily deduce further suitable genes encoding polypeptides having trans-selective BCO activity as defined herein which could be used for the conversion of beta-carotene into trans-retinal with a conversion/enzyme activity in the range of at least about 5%.

Particularly, the present invention is directed to a process for identification of modified BCO enzymes as defined herein, i.e. BCO enzymes with increased trans-specificity but high activity as defined herein, said process comprising the steps of:
(1) alignment of different beta-carotene oxidase enzymes, including but not limited to enzymes originated from insects, preferably from Drosophila, such as e.g. identified via BLAST search against UNIREF/UNIPROT databases, with SEQ ID NO:1, wherein the selected enzymes show high activity towards retinal production, i.e. in the range of at least about 5%, such as e.g. at least 2-fold higher than the BCO of Danio rerio,
(2) identify the positions in the selected enzymes corresponding to amino residue 91 and/or 499 in the polypeptide according to SEQ ID NO:1,
(3) introduction of at least one or two amino acid substitution(s) on position(s) corresponding to amino acid residue(s) selected from 91, 499 and combinations thereof identified in SEQ ID NO:1 in the aligned sequences; and
(4) screening for trans-retinal activity in a carotenoid-producing host cell, preferably selected from Yarrowia or Saccharomyces, with conversion rates of at least about 78 to 100% towards formation of trans-retinoids including trans-retinal, whereby the activity of the enzyme, i.e. conversion of beta-carotene into retinal, is about to stay in the range of at least about 5%.

More particularly, the present invention is directed to a process for increasing the trans-selectivity in BCO enzymes as defined herein, i.e. BCO enzymes with at least about 60% identity to SEQ ID NO:1 and a selectivity for formation of cis-retinal from conversion of beta-carotene in the range of more than 22% based on total retinoids, said trans-selectivity being increased by at least about 7%, comprising the steps of:
(1) alignment of different beta-carotene oxidase enzymes, including but not limited to enzymes originated from insects, preferably from Drosophila, such as e.g. identified via BLAST search against UNIREF/UNIPROT databases, with SEQ ID NO:1, preferably said sequences being characterized by a partial amino acid sequence of at least 5 amino acid residues selected from G-C-E-T-P corresponding to position 496 to 500 in the polypeptide according to SEQ ID NO:1, wherein the selected enzymes show high activity towards retinal production, i.e. in the range of about 10%,
(2) identify the positions in the selected enzymes corresponding to amino residue 91 and/or 499 in the polypeptide according to SEQ ID NO:1,
(3) introduction of at least one or two amino acid substitution(s) on position(s) corresponding to amino acid residue(s) selected from 91, 499 and combinations thereof identified in SEQ ID NO:1 in the aligned sequences; and
(4) screening for trans-retinal activity in a carotenoid-producing host cell, preferably selected from Yarrowia or Saccharomyces, with conversion rates of at least about 78 to 100% towards formation of trans-retinoids including trans-retinal.

The present invention is particularly directed to the use of such novel modified BCO enzymes, in a process for production of trans-retinal, wherein the production of cis-isoforms, such as e.g. cis-retinal, is reduced. The process might be performed with a suitable carotenoid or retinoid-producing host cell, particularly fungal host cell, expressing said modified BCO enzyme, preferably wherein the genes encoding said modified enzymes are heterologous expressed, i.e. introduced into said host cells. Retinal can be further converted into vitamin A by the action of (known) suitable chemical or biotechnological mechanisms, wherein the conversion of trans-isoforms, such as e.g. trans-retinal, into vitamin A is preferred.

Thus, the present invention is directed to a process for production of retinoids including a retinal-mix comprising trans-retinal in a percentage of at least about 78 to 100% based on the total retinals/retinoids produced by the host cell via enzymatic activity of a modified BCO enzyme as defined herein, comprising contacting beta-carotene with said modified BCO enzyme, and optionally isolating and/or purifying the formed trans-isoforms from the host cell or, which is the preferred way, further converting the retinal mix comprising at least about 78% of trans-retinal via enzymatic conversion into retinol and optionally into retinyl acetate with the same trans-ratio of about 78 to 100% based on total retinoids.

Particularly, the invention is directed to a process for production of vitamin A, said process comprising:
(a) introducing a nucleic acid molecule encoding one of the modified BCO enzymes as defined herein into a suitable carotenoid-producing host cell, particularly fungal host cell, as defined herein,
(b) enzymatic conversion, i.e. stereo-selective oxidation, of beta-carotene via action of said expressed modified BCO into at least about 78% of trans-retinal based on total retinoids,
(c) optionally, enzymatic conversion of retinal with a percentage of at least about 78% trans-retinal into retinol via action of retinol dehydrogenases,
(d) optionally, enzymatic conversion, i.e. acetylation, of retinol via action of acetyl transferase enzymes; and
(e) optionally, conversion of said retinyl acetate into vitamin A under suitable conditions known to the skilled person.

The terms “sequence identity”, “% identity” or “sequence homology” are used interchangeable herein. For the purpose of this invention, it is defined here that in order to determine the percentage of sequence homology or sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, Longden and Bleasby, Trends in Genetics 16, (6) pp 276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids, they are identical or have 100% identity.

The modified BCOs as defined herein also encompass enzymes carrying amino acid substitution(s) which do not alter enzyme activity, i.e. which show the same properties with respect to the enzymes defined herein and catalyze the conversion of beta-carotene into trans-retinal with conversion ratios of at least about 75 to 100% based on total retinoids including retinal, retinol, retinyl acetate. Such mutations are also called “silent mutations”, i.e. mutations which do not alter the (enzymatic) activity of the enzymes according to the present invention.

Expression of the enzymes/polynucleotides encoding one of the modified BCO enzymes as defined herein can be achieved in any host system, including (micro)organisms, which is suitable for retinoid (including retinal) production and which allows expression of the nucleic acids encoding one of the enzymes as disclosed herein, including functional equivalents or derivatives as described herein. Examples of suitable carotenoid-producing host (micro)organisms are bacteria, algae, fungi, including yeasts, plant or animal cells. Preferred bacteria are those of the genera Escherichia, such as, for example, Escherichia coli, Streptomyces, Pantoea (Erwinia), Bacillus, Flavobacterium, Synechococcus, Lactobacillus, Corynebacterium, Micrococcus, Mixococcus, Brevibacterium, Bradyrhizobium, Gordonia, Dietzia, Muricauda, Sphingomonas, Synochocystis, Paracoccus, such as, for example, Paracoccus zeaxanthinifaciens. Preferred eukaryotic microorganisms, in particular fungi including yeast, are selected from Saccharomyces, such as Saccharomyces cerevisiae, Aspergillus, such as Aspergillus niger, Pichia, such as Pichia pastoris, Hansenula, such as Hansenula polymorpha, Kluyveromyces, such as Kluyveromyces lactis, Phycomyces, such as Phycomyces blahesleanus, Mucor, Rhodotorula, Sporobolomyces, Xanthophyllomyces, Phaffia, Blakeslea, such as e.g. Blakeslea trispora, or Yarrowia, such as Yarrowia lipolytica. In particularly preferred is expression in a fungal host cell, such as e.g. Yarrowia or Saccharomyces, or expression in Escherichia, more preferably expression in Yarrowia lipolytica or Saccharomyces cerevisiae.

Depending on the host cell, the polynucleotides as defined herein for stereo-selective (i.e. trans-selective) formation of retinal with at least 75 to 100% as trans-retinal might be optimized for expression in the respective host cell. The skilled person knows how to generate such modified polynucleotides. It is understood that the polynucleotides as defined herein also encompass such host-optimized nucleic acid molecules as long as they still express the polypeptide with the respective activities as defined herein.

Thus, in one embodiment, the present invention is directed to a carotenoid-producing host cell, particularly fungal host cell, comprising polynucleotides encoding modified BCOs as defined herein which are optimized for expression in said host cell. Particularly, a carotenoid/retinoid-producing host cell, particularly fungal host cell, is selected from yeast, e.g. Yarrowia or Saccharomyces, such as Yarrowia lipolytica or Saccharomyces cerevisiae, wherein the polynucleotides encoding the modified BCOs as defined herein are selected from polynucleotides expressing modified polypeptides comprising at least one or two amino acid substitution(s) as defined herein in a sequence with at least about 60%, such as e.g. 65, 70, 75, 80, 85, 90, 92, 95, 97, 98, 99% or up to 100% identity to SEQ ID NO:1, such as e.g. introduction of amino acid substitution(s) at position(s) corresponding to residue 91 and/or 499 as defined herein in the polypeptide according SEQ ID NO:1.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code).

The present invention is directed to a process for production of retinal, in particular trans-isoform of retinal with an amount of at least 78% of trans-retinal, via enzymatic conversion of beta-carotene by the action of a modified BCO as described herein, wherein the said enzymes are preferably heterologous expressed in a suitable host cell under suitable conditions as described herein. The produced retinal, in particular trans-retinal, might be isolated and optionally further purified from the medium and/or host cell. In a further embodiment, retinal, in particular trans-retinal, can be used as precursor or building block in a multi-step process leading to vitamin A, such process comprising further conversion into retinol with further conversion/acetylation into retinyl acetate as known to the skilled person. Vitamin A might be isolated and optionally further purified from the medium and/or host cell as known in the art.

Compared to a process using a non-modified BCO as defined herein, the percentage of trans-retinoids, such as trans-retinal, can be increased by at least about 7%, such as in the range of about 7 to 33% or more, using a carotenoid/retinoid-producing host cell comprising/expressing one of the modified BCO-enzymes as defined herein, whereby the activity of the enzyme, i.e. conversion of beta-carotene into retinal, might stay about the same level, i.e. in the range of at least about 5%. Preferably, the host cell might be a fungal host cell, such as e.g. selected from Yarrowia or Saccharomyces.

The host cell, i.e. microorganism, algae, fungal, animal or plant cell, capable of expressing the beta-carotene producing genes, the modified BCOs as defined herein and/or optionally further genes required for biosynthesis of vitamin A, may be cultured in an aqueous medium supplemented with appropriate nutrients under aerobic or anaerobic conditions and as known by the skilled person for the different host cells. Optionally, such cultivation is in the presence of proteins and/or co-factors involved in transfer of electrons, as defined herein. The cultivation/growth of the host cell may be conducted in batch, fed-batch, semi-continuous or continuous mode. Depending on the host cell, preferably, production of retinoids such as e.g. vitamin A and precursors such as retinal, retinol, and/or retinyl acetate can vary, as it is known to the skilled person. Cultivation and isolation of beta-carotene- and retinoid-producing host cells selected from Yarrowia and Saccharomyces is described in e.g. WO2008042338 or WO2014096992. With regards to production of beta-carotene and retinoids in E. coli as host cell, methods are described in e.g. US20070166782.

As used herein, a “carotenoid-producing host cell”, particularly fungal or bacterial host cell, is a host cell, wherein the respective polypeptides are expressed and active in vivo leading to production of carotenoids, e.g. beta-carotene. The genes and methods to generate carotenoid-producing host cells are known in the art, see e.g. WO2006102342. Depending on the carotenoid to be produced, different genes might be involved, such as e.g. genes encoding geranylgeranyl synthase, phytoene synthase, phytoene desaturase, lycopene cyclase as known in the art (such as e.g. as described in US20160130628 or WO2009126890).

As used herein, a “retinoid-producing host cell”, particularly fungal or bacterial host cell, is a host cell wherein the respective polypeptides are expressed and active in vivo, leading to production of retinoids, e.g. vitamin A and its precursors retinal and/or retinol, via enzymatic conversion of beta-carotene. These polypeptides include the modified BCOs as defined herein. The genes of the vitamin A pathway and methods to generate retinoid-producing host cells are known in the art: when transformed with modified BCO genes as described herein, retinal, with at least about 75% of trans-retinal based on total retinoids, can be produced. Optionally, when transformed with retinol dehydrogenase, then retinol can be produced. The retinol can optionally be acetylated by transformation with genes encoding alcohol acetyl transferases. Optionally, the endogenous retinol acylating genes can be deleted and/or inactivated. Further, optionally the enzymes can be selected to produce and acetylate the trans form of retinol to yield a high amount of all-trans retinyl acetate. The trans-specificity due to the modified BCO enzymes according to the present invention is similar and independent on the use of the host cell, such as retinoid-producing host cell, as e.g. using a fungal host cell including but not limited to Yarrowia lipolytica or Saccharomyces cerevisiae, with a percentage of at least about 5% conversion of beta-carotene into retinal.

Preferably, the beta-carotene is converted into retinal (with at least 78 to 100% as trans-retinal) via action of modified BCO as defined herein, the retinal is further converted into retinol via action of enzymes having retinol dehydrogenase activity, and the retinol is converted into retinol acetate via action of acetyl-transferase enzymes, such as e.g. ATF1. The retinol acetate might be the retinoid of choice which is isolated from the host cell.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a suitable (cell-free) system in the presence of a suitable substrate. The skilled person knows how to measure enzyme activity, in particular activity of modified BCOs as defined herein. Analytical methods to evaluate the capability of a suitable modified BCO as defined herein for trans-retinal production from conversion of beta-carotene are known in the art, such as e.g. described in Example 4 of WO2014096992. In brief, titers of products such as trans-retinal, cis-retinal, beta-carotene and the like can be measured by HPLC.

In one specific embodiment, the process according to the present invention is carried out using modified insect BCOs, particularly originated from Drosophila melanogaster, wherein at least 1% retinal is generated in 200 h corn oil fed fermentation with Yarrowia as host, wherein the BCO is expressed as single Tell promoter driven copy (DmNinaB). The activity of the insect BCOs (either non-modified or modified) is in the range of at least 2-fold higher than the activity of a BCO isolated from Danio rerio known from the database as NP_001315424.

“Retinoids” as used herein include beta-carotene cleavage products also known as apocarotenoids, including but not limited to retinal, retinolic acid, retinol, retinoic methoxide, retinyl acetate, retinyl esters, 4-keto-retinoids, 3 hydroxy-retinoids or combinations thereof. Biosynthesis of retinoids is described in e.g. WO2008042338.

“Retinal” as used herein is known under IUPAC name (2E,4E,6E,8E)-3,7-Dimethyl-9-(2,6,6-trimethylcyclohexen-1-yl)nona-2,4,6,8-tetraenal. It is herein interchangeably referred to as retinaldehyde or vitamin A aldehyde and includes both cis- and trans-isoforms, such as e.g. 11-cis retinal, 13-cis retinal, trans-retinal and all-trans retinal. A mixture of cis- and trans-retinal is referred to herein as “retinal mix”, wherein the percentage of “at least about 78%” with regards to trans-retinal or “about 22% or less” with regards to cis-retinal refers to the ratio of trans-retinal or cis-retinal in such retinal mix based on total retinoids in the mix. A ratio of up to 22% of cis-retinal based on total retinoids obtained via enzymatic conversion of beta-carotene is referred herein as “relatively high percentage of cis-selectivity” and which is to be reduced by using modified BCO enzymes as defined herein. Due to instability of retinal, trans- and cis-specificity is often measured in intermediates such as e.g. retinol (which is the direct product from conversion of retinal via RDH) or retinyl acetate (which is the direct product from conversion of retinol via ATF1).

The term “carotenoids” as used herein is well known in the art. It includes long, 40 carbon conjugated isoprenoid polyenes that are formed in nature by the ligation of two 20 carbon geranylgeranyl pyrophosphate molecules. These include but are not limited to phytoene, lycopene, and carotene, such as e.g. beta-carotene, which can be oxidized on the 4-keto position or 3-hydroxy position to yield canthaxanthin, zeaxanthin, or astaxanthin. Biosynthesis of carotenoids is described in e.g. WO2006102342.

“Vitamin A” as used herein may be any chemical form of vitamin A found in aqueous solutions, in solids and formulations, and includes retinol, retinyl acetate and retinyl esters. It also includes retinoic acid, such as for instance undissociated, in its free acid form or dissociated as an anion.

The following examples are illustrative only and are not intended to limit the scope of the invention in any way. The contents of all references, patent applications, patents and published patent applications, cited throughout this application are hereby incorporated by reference, in particular WO2006102342, WO2008042338 or WO2014096992.

### EXAMPLES

### Example 1: General Methods, Strains and Plasmids

All basic molecular biology and DNA manipulation procedures described herein are generally performed according to Sambrook et al. (eds.), Molecular Cloning: A Laboratory Manual. Cold Spring Harbor Laboratory Press: New York (1989) or Ausubel et al. (eds). Current Protocols in Molecular Biology. Wiley: New York (1998).

Shake plate assay. Typically, 800 μl of 0.075% Yeast extract, 0.25% peptone (0.25× YP) is inoculated with 10 μl of freshly grown Yarrowia and overlaid with 200 μl of Drakeol 5 mineral oil carbon source 5% corn oil in mineral oil and/or 5% in glucose in aqueous phase. Transformants were grown in 24 well plates (Multitron, 30° C., 800 RPM) in YPD media with 20% dodecane for 4 days. The mineral oil fraction was removed from the shake plate wells and analyzed by HPLC on a normal phase column, with a photo-diode array detector.

DNA transformation. Strains are transformed by overnight growth on YPD plate media 50 μl of cells is scraped from a plate and transformed by incubation in 500 μl with 1 μg transforming DNA, typically linear DNA for integrative transformation, 40% PEG 3550MW, 100 mM lithium acetate, 50 mM Dithiothreitol, 5 mM Tris-Cl pH 8.0, 0.5 mM EDTA for 60 minutes at 40° C. and plated directly to selective media or in the case of dominant antibiotic marker selection the cells are out grown on YPD liquid media for 4 hours at 30° C. before plating on the selective media.

DNA molecular biology. Genes were synthesized with NheI and MluI ends in pUC57 vector. Typically, the genes were subcloned to the MB5082 ‘URA3’, MB6157 HygR, and MB8327 Nat® vectors for marker selection in Yarrowia lipolytica transformations, as in WO2016172282. For clean gene insertion by random nonhomologous end joining of the gene and marker HindIII/XbaI (MB5082) or PvulI (MB6157 and MB8327), respectively purified by gel electrophoresis and Qiagen gel purification column.

Plasmid list. Plasmid, strains and codon-optimized sequences to be used are listed in Table 1, 2 and the sequence listing. Nucleotide sequence ID NO:2 is codon optimized for expression in Yarrowia.

TABLE 1
list of plasmids used for construction of the strains carrying
the heterologous modified BCO-genes. The sequence ID
NOs refer to the inserts. For more details, see text.

| | | | SEQ ID NO: |
|---|---|---|---|
| MB plasmid | Backbone MB | Insert | (aa/nt) |
| 8457 | 5082 | DmBCO | ½ |

TABLE 2
list of Yarrowia strains used for production of retinoids carrying the
heterologous (non-modified or modified) BCO genes. For more details, see text.

| ML strain | Description | First described in |
|---|---|---|
| 7788 | Carotene strain | WO2016172282 |
| 15710 | ML7788 transformed with MB7311-Mucor CarG | WO2016172282 |
| 17544 | ML15710 cured of URA3 by FOA and HygR by | here |
| | Cre/lox | |
| 17767 | ML17544 transformed with MB6072 DmBCO-URA3 | here |
| | and MB6732 SbATF1-HygR and cured of markers | |
| 17978 | ML17968 transformed with MB8200 FfRDH-URA3 | here |
| | and cured of markers | |

Normal phase retinol method. A Waters 1525 binary pump attached to a Waters 717 auto sampler were used to inject samples. A Phenomenex Luna 3μ Silica (2), 150×4.6 mm with a security silica guard column kit was used to resolve retinoids. The mobile phase consists of either, 1000 mL hexane, 30 mL isopropanol, and 0.1 mL acetic acid for astaxanthin related compounds, or 1000 mL hexane, 60 mL isopropanol, and 0.1 mL acetic acid for zeaxanthin related compounds. The flow rate for each is 0.6 mL per minute. Column temperature is ambient. The injection volume is 20 μL. The detector is a photodiode array detector collecting from 210 to 600 nm. Analytes were detected according to Table 3.

TABLE 3
list of analytes using normal phase retinol method. The
addition of all added intermediates gives the amount
of total retinoids. For more details, see text.

| | Retention | Lambda |
|---|---|---|
| Intermediates | time [min] | max [nm] |
| 11-cis-dihydro-retinol | 7.1 | 293 |
| 11-cis-retinal | 4 | 364 |
| 11-cis-retinol | 8.6 | 318 |
| 13-cis-retinal | 4.1 | 364 |
| dihydro-retinol | 9.2 | 292 |
| retinyl-acetate | 3.5 | 326 |
| retinyl-ester | 3 | 325 |
| trans-retinal | 4.7 | 376 |
| trans-retinol | 10.5 | 325 |

Sample preparation. Samples were prepared by various methods depending on the conditions. For whole broth or washed broth samples the broth was placed in a Precellys® tube weighed and mobile phase was added, the samples were processed in a Precellys® homogenizer (Bertin Corp, Rockville, Md., USA) on the highest setting 3× according to the manufactures directions. In the washed broth the samples were spun in a 1.7 ml tube in a microfuge at 10000 rpm for 1 minute, the broth decanted, 1 ml water added mixed pelleted and decanted and brought up to the original volume the mixture was pelleted again and brought up in appropriate amount of mobile phase and processed by Precellys® bead beating. For analysis of mineral oil fraction, the sample was spun at 4000 RPM for 10 minutes and the oil was decanted off the top by positive displacement pipet (Eppendorf, Hauppauge, N.Y., USA) and diluted into mobile phase mixed by vortexing and measured for retinoid concentration by HPLC analysis.

Fermentation conditions. Fermentations (especially on larger scale) were identical to the previously described conditions using mineral oil overlay and stirred tank that was corn oil fed in a bench top reactor with 0.5 L to 5 L total volume (see WO2016172282). Generally, the same results were observed with a fed batch stirred tank reactor with an increased productivity demonstrating the utility of the system for the production of retinoids.

### Example 2: Production of Trans-Retinal in Yarrowia lipolytica

Typically, a beta carotene strain ML17544 was transformed with purified linear DNA fragment by HindII and XbaI mediated restriction endonucleotide cleavage of beta carotene oxidase (non-modified or modified BCO) containing codon optimized fragments linked to a URA3 nutritional marker. Transforming DNA were derived from MB6702 Drosophila NinaB BCO gene, whereby the codon-optimized sequence (SEQ ID NO:2) had been used. The genes were then grown screening 6-8 isolates in a shake plate analysis, and isolates that performed well were run in a fed batch stirred tank reaction for 8-10 days. Detection of cis-and trans-retinal was made by HPLC using standard parameters as described in WO2014096992, but calibrated with purified standards for the retinoid analytes. The amount of trans-retinal in the retinal mix could be increased to at least 78.1 up to 96.5% using the modified BCOs. The wild-type, i.e. non-modified, BCO from Drosophila melanogaster (SEQ ID NO:1) resulted in only 73% of trans-retinal based on total retinoids (see Table 4). Further, a native RDH reduces retinal to retinol in Yarrowia lipolytica. These isomers can also be monitored as surrogates for the retinal cis/trans isomers. The enzyme activity indicating the generation of retinal from conversion of beta-carotene was about the same irrespectively whether the wildtype or modified BCOs were used (about 5 to 20% conversion into retinal).

TABLE 4
Retinal production in Yarrowia as enhanced by action of modified
BCOs originated from Drosophila melanogaster (DmNinaB).
“% trans” means percentage of trans-retinal in the mix of retinoids;
“DCW” means dry cell weight”. For more details, see text.

| | | % | | |
|---|---|---|---|---|
| | % | retinoids/ | ML | MB |
| BCO gene | trans- | DCW | strain | plasmid |
| DmNinaB wt | 73 | 14 | 17544 | 6702 |
| DmNinaB T499L | 95.3 | 9.8 | 17544 | 9343 |
| DmNinaB L91W-T499L | 96.5 | 7.3 | 17544 | 9357 |
| DmNinaB L91F-T499L | 91.7 | 7.6 | 17544 | 9358 |
| DmNinaB T499M | 96.4 | 9.9 | 17544 | 9360 |
| DmNinaB T4991 | 83.2 | 5.2 | 17544 | 9363 |
| DmNinaB L91F | 90.8 | 14 | 17544 | 9339 |
| DmNinaB L91W | 78.1 | 14 | 17544 | 9338 |

Furthermore, various insect BCOs with at least about 60% identity to SEQ ID NO:1 were tested for occurrence of the amino acid residues on positions corresponding to L91, L336, M364, T499, and L611 (see Table 5).

Surrounding amino acids were identified by modeling the structure of the enzyme encoded by SEQ ID NO:1 using the software program Yasara (https://www.yasara.org/) using the following parameters and PDB code 4RSC (downloadable from http://www.pdb.org) as the template structure: Modeling speed (slow=best): Slow

Number of PSI-BLAST iterations in template search (PsiBLASTs): 3

Maximum allowed (PSI-)BLAST E-value to consider template (EValue Max): 0.5

Maximum number of templates to be used (Templates Total): 1

Maximum number of templates with same sequence (Templates SameSeq): 1

Maximum oligomerization state (OligoState): 4 (tetrameric)

Maximum number of alignment variations per template: (Alignments): 3

Maximum number of conformations tried per loop (LoopSamples): 50

Maximum number of residues added to the termini (TermExtension): 10

The homology model that is produced by Yasara can subsequently be inspected by someone skilled in the art to identify residues surrounding the mutated positions 91 and 499. Subsequently, an alignment was made from the closest homologous sequences from the Uniref/Swissprot database (https://www.uniref.org) that score 58% and up compared to SEQ ID NO:1, and the conservancy of the 5 positions above is checked. This data is shown in Table 5. All residues are strictly conserved of the mutated positions 91 and 499 and their surrounding residues 336, 364 and 611, which are directly in the active site where the beta-carotene substrate binds and close to the metal ion bound by the conserved catalytic His cluster in the enzyme, and therefore it is expected that the effect of the claimed mutations on cis/trans-specificity will also be the same in these homologous sequences.

TABLE 5
Blast search for insect BCOs with at least 60% identity to SEQ ID NO: 1 showing conserved
amino acids corresponding to position 91, 499, 336, 364 and 611. The “reference #”
is the UNIREF-SWISSPROT database code (www.uniprot.org), “identity” is the longest
identity to DmBCO1 (SEQ ID NO: 1), “L91” means corresponding AA on 91L mutation
position, “T499” means corresponding AA on 499T mutation position, “L336”
means corresponding AA on 336L surrounding position, “M364” means corresponding
AA on 364M surrounding position, and “L611” means corresponding AA on 611L surrounding
position. The molecular function annotation for all shown sequences is beta-carotene
15,15′-monooxygenase. For more details, see text.

| | | Identity | | | | | |
|---|---|---|---|---|---|---|---|
| Reference # | Organism | [%] | L91 | T499 | L336 | M364 | L611 |
| SEQ ID NO: 1 | Drosophila melanogaster | 100 | L | T | L | M | L |
| B3P415 | Drosophila erecta | 96.0% | L | T | L | M | L |
| A0A1W4V4X0 | Drosophila ficusphila | 93.6% | L | T | L | M | L |
| B3LWV8 | Drosophila ananassae | 90.2% | L | T | L | M | L |
| B4G3J1 | Drosophila persimilis | 84.5% | L | T | L | M | L |
| Q299Z6 | Drosophila pseudoobscura pseudoobscura | 84.4% | L | T | L | M | L |
| B4NH74 | Drosophila willistoni | 84.0% | L | T | L | M | L |
| A0A3B0K9S8 | Drosophila guanche | 83.9% | L | T | L | M | L |
| B4JYB7 | Drosophila grimshawi | 83.1% | L | T | L | M | L |
| B4KBR0 | Drosophila mojavensis | 83.1% | L | T | L | M | L |
| A0A3B0K2Y2 | Drosophila guanche | 82.3% | L | T | L | M | L |
| A0A0M3QXZ7 | Drosophila busckii | 82.3% | L | T | L | M | L |
| B4MBL2 | Drosophila virilis | 81.4% | L | T | L | M | L |
| A0A0Q9WZH1 | Drosophila mojavensis | 81.3% | L | T | L | M | L |
| W8AFR2 | Ceratitis capitata | 71.8% | L | T | L | M | L |
| A0A1A9V8V6 | Glossina austeni | 65.1% | L | T | L | M | L |
| A0A1A9XM81 | Glossina fuscipes fuscipes | 64.7% | L | T | L | M | L |
| A0A1B0FGT0 | Glossina morsitans morsitans | 64.4% | L | T | L | M | L |
| A0A1B0BNS4 | Glossina palpalis gambiensis | 64.2% | L | T | L | M | L |
| A0A182NUF8 | Anopheles dirus | 61.5% | L | T | L | M | L |
| A0A182Y0A2 | Anopheles stephensi | 61.3% | L | T | L | M | L |
| A0A182WU08 | Anopheles quadriannulatus | 61.2% | L | T | L | M | L |
| A0A182URE3 | Anopheles merus | 60.9% | L | T | L | M | L |
| A0A182PPK6 | Anopheles epiroticus | 60.7% | L | T | L | M | L |
| A0A182W715 | Anopheles minimus | 60.5% | L | T | L | M | L |
| A0A182LRC9 | Anopheles culicifacies | 60.4% | L | T | L | M | L |
| A0A182JU85 | Anopheles christyi | 60.2% | L | T | L | M | L |
| A0A182F4X0 | Anopheles albimanus | 60.2% | L | T | L | M | L |
| A0A1Y9GLE5 | Anopheles arabiensis | 60.2% | L | T | L | M | L |
| A0A182L1F0 | Anopheles coluzzii | 60.1% | L | T | L | M | L |
| A0A182Q6V3 | Anopheles farauti | 60.0% | L | T | L | M | L |
| A0A182SJU9 | Anopheles maculatus | 59.5% | L | T | L | M | L |
| W5J3D8 | Anopheles darlingi | 59.0% | L | T | L | M | L |
| A0A182H5Q0 | Aedes albopictus | 58.7% | L | T | L | M | L |
| Q17FY3 | Aedes aegypti | 58.5% | L | T | L | M | L |

### Example 3: Production of Trans-Retinal in Saccharomyces cerevisiae

Typically, a beta carotene strain is transformed with heterologous genes encoding for enzymes such as geranylgeranyl synthase, phytoene synthase, lycopene synthase, lycopene cyclase constructed that is producing beta carotene according to standard methods as known in the art (such as e.g. as described in US20160130628, WO2009126890 or Verwaal et al., Applied and Environmental Microbiology, Vol. 73, No. 13, pp. 4342-4350, 2007). Carotene producing strain MY4378 (CEN.PK113-7D FBA1p-crtE; TEF1p-crtYB; ENO1p-crtI) is transformed with modified BCOs that are codon optimized for expression in Saccharomyces like vector MB8433 (DmNinaB wt HYGR) to make strain MY4382(CEN.PK113-7D FBA1p-crtE; TEF1p-crtYB; ENO1p-crtI TEF1p-DmNinaB wt HYGR) or in an analogous fashion to result in at least 78% trans-retinal based on total retinoids including retinal. Optionally, when transformed with retinol dehydrogenase from vector MB8431, then retinol can be produced. Vector MB8433 is constructed as an integrating Hygromycin selectable vector based on the backbone MB7622 (SEQ ID NO:3) by insertion of the coding sequence into the unique BamHI/EcoRI sites to yield vectors MB8431 (SEQ ID NO:4) and MB8433 (SEQ ID NO:5). Further, optionally the enzymes can be selected to produce and acetylate the trans form of retinol to yield a high amount of all-trans retinyl acetate.

Using the BCOs according to Table 4 in S. saccharomyces as host cell, conversion of beta-carotene into retinal with percentage of at least about 5% can be obtained, with a selectivity for trans-retinal based on total retinoids in the range of at least about 78%. The % retinoids/DCW is much lower as compared to Yarrowia lipolytica as host cell, such as e.g. in the range of about 2 to 3 (data not shown).

1. A beta-carotene oxidase enzyme, preferably insect enzyme, more preferably enzyme originated from Drosophila, comprising one or more amino acid substitution(s) in a sequence with at least about 60%, such as 65, 70, 75, 80, 85, 90, 92, 95, 97, 98, 99% or up to 100% identity to SEQ ID NO:1, wherein the one or more amino acid substitution(s) are located at position(s) corresponding to amino acid residue(s) selected from 91 and/or 499 in the polypeptide according to SEQ ID NO:1 and wherein the amino acid of residue 91 being tryptophan or phenylalanine and/or amino acids of residue 499 being selected from methionine, leucine or isoleucine.

2. The enzyme according to claim 1 catalyzing the conversion of beta-carotene into retinal with a ratio of at least about 78% as trans-retinal based on total retinoids.

3. The enzyme according to claim 1, wherein at least about 5% of beta-carotene is converted into retinal.

4. The enzyme according to claim 1, wherein the specificity towards trans-isoforms including the formation of trans-retinal is increased by at least about 7% based on total retinoids compared to the trans-specificity of the respective enzyme without carrying one or more of said amino acid substitution(s).

5. The enzyme according to claim 1, comprising a single amino acid substitution located at a position corresponding to amino acid residues selected from 91 and/or 499, preferably selected from residue 499, in the polypeptide according to SEQ ID NO:1.

6. The enzyme according to claim 1, comprising at least two amino acid substitutions at positions corresponding to amino acid residues selected from 91 and 499 in the polypeptide according to SEQ ID NO:1.

7. The enzyme according to claim 1 which is expressed in a carotenoid-producing host cell, preferably a fungal host cell, more preferably selected from Yarrowia or Saccharomyces.

8. A carotenoid-producing host cell, particularly fungal host cell, comprising an enzyme according to claim 1, wherein said host cell being preferably selected from Yarrowia or Saccharomyces and being transformed with a polynucleotide expressing said enzyme.

9. A process for production of trans-retinal comprising providing a carotenoid-producing host cell according to claim 8, cultivating said host cell in a suitable culture medium under suitable culture conditions, and optionally isolating and/or purifying the trans-retinal from the medium, wherein the ratio of trans-retinal is in the range of at least about 78% based on total retinoids.

10. A process for increasing the conversion of beta-carotene into trans-retinal by at least 7% based on total retinoids in a carotenoid-producing host cell comprising transforming said host cell, preferably fungal host cell, more preferably a host cell selected from Yarrowia or Saccharomyces, with an enzyme according to claim 1.

11. A process for production of vitamin A comprising the steps of:
(a) introducing a nucleic acid molecule encoding one of the modified BCO enzymes according to claim 1 into a suitable carotenoid-producing host cell, particularly fungal host cell,
(b) enzymatic conversion, i.e. stereo-selective oxidation, of beta-carotene via action of said expressed modified BCO into at least about 78% of trans-retinal based on total retinoids,
(c) optionally, enzymatic conversion of retinal with a percentage of at least about 75% trans-retinal into retinol via action of retinol dehydrogenases,
(d) optionally, enzymatic conversion, i.e. acetylation, of retinol via action of acetyl transferase enzymes; and
(e) optionally, conversion of said retinyl acetate into vitamin A under suitable conditions known to the skilled person.

12. Use of an enzyme according to claim 1 in a process for production of retinyl acetate in a suitable host cell, comprising the step of conversion of beta-carotene into retinal by the action of said enzyme and optionally further enzymatic conversion into retinyl acetate.

13. Use according to claim 12, wherein the percentage of trans retinyl acetate is in the range of at least about 78% based on total retinoids.

14. Method for increasing the trans-specificity of a beta-carotene oxidase enzyme comprising the steps of:
(1) alignment of different beta-carotene oxidase enzymes, including but not limited to enzymes originated from insects, preferably from Drosophila, such as e.g. identified via BLAST search against UNIREF/UNIPROT databases, with SEQ ID NO:1, wherein the selected enzymes show high activity towards retinal production, i.e. in the range of at least about 5%, such as e.g. at least 2-fold higher than the BCO of Danio rerio,
(2) identification of the positions in the selected enzymes corresponding to amino residue 91 and/or 499 in the polypeptide according to SEQ ID NO:1,
(3) introduction of at least one or two amino acid substitution(s) on position(s) corresponding to amino acid residue(s) selected from 91, 499 and combinations thereof identified in SEQ ID NO:1 in the aligned sequences; and
(4) screening for trans-retinal activity in a carotenoid-producing host cell, preferably selected from Yarrowia or Saccharomyces, with conversion rates of at least about 78 to 100% towards formation of trans-retinal based on total retinoids, whereby the activity of the enzyme, i.e. conversion of beta-carotene into retinal, is in the range of at least about 5%.
